(19) United States
(12) Patent Application Publication
(10) Pub. No.: US 2004/0003423 A1
(43) Pub. Date: Jan. 1, 2004

(54) Transgenic knockout animal model null for pigment epithelium-derived factor (PEDF)

(75) Inventors: Noel P. Bouck, Occidental, CA (US); Susan E. Crawford, Burr Ridge, IL (US); Veronica Stellmach, Chicago, IL (US)

Correspondence Address: MEDLEN & CARROLL, LLP, Suite 350, 101 Howard Street, San Francisco, CA, 94105, US

(73) Assignee: Northwestern University, Evanston, IL

(21) Appl. No.: 10/361,516

(22) Filed: Feb. 10, 2003

Related U.S. Application Data
(60) Provisional application No. 60/355,222, filed on Feb. 8, 2002.

(51) Int. Cl. A01K 67/027; A61K 49/00

(52) U.S. Cl. 800/18; 800/3

(57) ABSTRACT

The present invention relates to transgenic knockout animal models null for pigment epithelium-derived factor (PEDF). The present invention also provides methods for generating animal disease models and screening methods for identifying biologically active compounds.

The present Application claims priority to U.S. Provisional Application No. 60/355,222 filed Feb. 8, 2002, herein incorporated by reference.

The present invention was made in part with funding under National Cancer Institute Grant Nos. CA52750-08 and CA64239-08. The government may have certain rights in the invention.

### FIELD OF THE INVENTION

The present invention relates to transgenic knockout animal models null for pigment epithelium-derived factor (PEDF). The present invention also provides methods for generating animal disease models and screening methods for identifying biologically active compounds.

### BACKGROUND

With the near completion of the human genome and the genomes of other higher animals, a wealth of genetic information has become available. However, this sequence information alone is not sufficient to provide an understanding of the tens to hundreds of thousands of complex biological reaction that define an organism's health and metabolism. Most of the work conducted on understanding gene expression to date has utilized in vitro studies. Unfortunately, much of the information obtained by these studies does not provide sufficient information to understand the molecular mechanisms of health in vivo. What are needed are new systems for studying biological systems in vivo.

### SUMMARY OF THE INVENTION

The present invention relates to transgenic knockout animal models null for pigment epithelium-derived factor (PEDF). The present invention also provides methods for generating animal disease models and screening methods for identifying biologically active compounds.

In some embodiments, the present invention provides nonhuman animals with somatic and germ cells having a functional disruption of at least one, and more preferably both, alleles of an endogenous PEDF gene. Accordingly, the invention provides viable animals having a mutated or deleted PEDF gene, and thus lacking PEDF activity. These animals are characterized in that they have hyperplasia of the prostate with stratification of nuclei, nuclear pleomorphism and an increased nuclear to cytoplasmic ratio. The prostate epithelial cells show a higher proliferative index. Also, the stroma shows a change in microvascular density. These animals also show a disturbance in the ganglia cells of the retina with layering of retinal photoreceptors. The optic nerve is also smaller than wild-type animals. The animals further show a drop-out in cerebellar granule cells. The animals also show a change in kidney vasculature with an approximately 2-fold higher than baseline secretion of VEG-F. These animals also show increase microvascular density in the prostate, pancreas, kindney and retina. Finally, these animals also show an enlarged pancreas, with acinar epithelial cells appearing less well differentiated with increased nuclear to cytoplasmic ratios and cytologic atypia.

Any method for generating knockout animals is contemplated by the present invention. In some embodiments, the PEDF gene preferably is disrupted by homologous recombination between the endogenous allele and a mutant PEDF gene or appropriate sequence to delete the endogenous allele, or portion thereof, that has been introduced into an embryonic stem cell precursor of the animal. The embryonic stem cell precursor is then allowed to develop, resulting in an animal having a functionally disrupted PEDF gene. The animal may have one PEDF gene allele functionally disrupted (i.e., the animal may be heterozygous for the null mutation), or more preferably, the animal has both PEDF gene alleles functionally disrupted (i.e., the animal can be homozygous for the mutation). In one embodiment of the invention, functional disruption of both PEDF gene alleles produces animals in which expression of the PEDF gene product in cells of the animal is substantially or completely absent relative to non-mutant animals. In another embodiment, the PEDF gene alleles are disrupted such that an altered (i.e., mutant) PEDF gene product is produced in cells of the animal. A preferred nonhuman animal of the invention having a functionally disrupted PEDF gene is a mouse.

In certain embodiments, the present invention provides methods of generating a transgenic animal, comprising crossing the knockout animal of the present invention (e.g. a mouse) with a second animal (e.g. a second mouse). In some embodiments, the second animal is selected from the group consisting of a p27 knockout animal, a PTEN knockout animal, a thrombospondin knockout animal, and a p53 knockout animal.

In some embodiments, the present invention provides methods of screening a compound (agent), comprising: a) exposing the PEDF knockout animal to a compound; and b) determining a response of the animal to the compound. In certain embodiments, a change in response compared to a PEDF knockout animal not exposed to the compound, indicates the response to the compound. In other embodiments, the animals (cells, tissue or organs of the animal) are examined directly without comparison to a wild-type animal.

In certain embodiments, the compound tested is a candidate anti-angiogenic compound (e.g. a compound hoped to have anti-angiogenic properties). In particular embodiments, the compound tested is a known anti-angiogenic compound (e.g. a statin). In some embodiments, in determining the response the animal has to the compound, an organ or tissue selected from: retinal, retinal tissue, kidney, kidney tissue, pancreas pancreatic tissue, prostate, prostatic tissue, bladder, bladder tissue, or combinations thereof, are examined. Compounds that, for example, reduce or prevent excessive microvascular density in these organs or tissues may be considered potentially anti-angiogenic. In certain embodiments, tumors are present (or induced) on the PEDF knockout animals. In certain embodiments, tumors are composed of pancreatic, prostate, bladder, kidney, or retinal cells. In further embodiments, the compound (test compound) is administered to the tumor on the knockout animal to screen for anti-angiogenic, anti-tumor compounds.

In some embodiments, the compound tested is a candidate carcinogen or a known carcinogen. In certain embodiments, compounds that have been screened in other animal models, and been determined to have low or no carcinogenic activity, are screened in the animal models of the present invention. While not limited to any mechanism, it is believed that the animal models of the present invention have an increased susceptibility to carcinogenic compounds. Again, while not limited to any mechanism, it is believed that the lack of PEDF expression (or reduced PEDF expression) makes the animal models of the present invention particularly susceptible to developing cancer when exposed to carcinogens. As such, the animal models of the present invention allow the carcinogenic potential of candidate compounds to be readily evaluated.

In other embodiments, the present invention provides methods for inducing cancer in a transgenic knockout animal (e.g. mouse, rat, pig, chimpanzee, etc.), comprising; a) providing a transgenic knockout animal whose genome comprises a homozygous or heterozygous disruption in its endogenous pigment epithelium-derived factor gene, wherein the homozygous disruption prevents the expression of a functional pigment eptithelium-derived factor protein, and wherein the heterozygous disruption reduces the expression of functional pigment eptithelium-derived factor protein in the knockout animal; and b) exposing the transgenic knockout mouse to conditions such that cancer formation is promoted in the transgenic knockout animal.

In particular embodiments, the conditions comprise administering a carcinogen to the transgenic knockout animal or exposing the animal to high levels of stress. In some embodiments, the conditions comprise injecting cancerous cells into the transgenic knockout animal (e.g. cancerous bladder cells, cancerous pancreatic cells, cancerous prostate cells, cancerous kidney cells, cancerous retinal cells, or combinations thereof). In other embodiments, the conditions comprise exposing the transgenic knockout animal to radiation or high levels of ultraviolet light. In certain embodiments, the conditions comprise exposing the transgenic knockout animal to biological agents known to induce cancer (e.g. certain viruses).

### DESCRIPTION OF THE FIGURES

FIG. 1A shows the eight exons of the murine PEDF gene, indicated by black rectangles on the 18.8 EcoRI (R) fragment, with the arrow indicating the translational start site. The sequence of a murine PEDF nucleic acid sequence is provided by Accession Nos. AF017057 and AF036164, both of which are herein incorporated by reference.

FIG. 1B shows a null allele construct, with exons 3-6 replaced with a Lac Z neomycin (Neo) resistance gene cassette. Lox P sites (triangles) border the Neo cassette and add an EcorRI site. An IRES site (stippled box) allows translation of the Lac Z gene driven by the PEDF promoter, and Neo expression is driven by the phosphoglycerate kinase promoter.

### DETAILED DESCRIPTION OF THE INVENTION

The present invention provides transgenic animals having somatic and germ cells in which at least one allele of an endogenous pigment epithelium-derived factor (PEDF) gene is functionally disrupted. Preferred embodiments of the present invention are illustrated below using the example of a PEDF null mouse model. The mouse may be heterozygous or, more preferably, homozygous for the PEDF gene disruption. As used herein, the term “gene disruption” refers to any heritable genetic alteration that prevents normal production of PEDF protein (e.g., prevents expression of a PEDF gene product, expression of normal PEDF gene product, or prevents expression of normal amounts of the PEDF gene product). In some embodiments, the gene disruption comprises a deletion of all or a portion of the PEDF gene. In other embodiments, the gene disruption comprises an insertion or other mutation of the PEDF gene. In still other embodiments, the gene disruption is a genetic alteration that prevents expression, processing, or translation of the PEDF gene. In one embodiment, both PEDF gene alleles are functionally disrupted such that expression of the PEDF gene product is substantially reduced or absent in cells of the animal. The term “substantially reduced or absent” is intended to mean that essentially undetectable amounts of normal PEDF gene product are produced in cells of the animal. This type of mutation is also referred to as a “null mutation” and an animal carrying such a null mutation is also referred to as a “knockout animal.”

PEDF is a 50 kDa secreted glycoprotein with potent neurotrophic and anti-angiogenic activities. PEDF can trigger neurite outgrowths in retinoblastoma cells (Tobran-Tink and Johnson, Invest. Ophthalmol. Visual Sic. 30:1700) and act as a survival factor for cerebellar granule cells (Taniwaki, et al., J. Neurochem 64:2509), spinal motor neurons (Bilak et al., J. Neuropath. Exp. Neurol. 58:719), and retinal neurons (Cao et al., J. Neurosci. Res. 57:789). It was recently discovered that PEDF also has potent anti-angiogenic activity (Dawson et al., Science 285:245). The effective dose of PEDF for inhibiting endothelial cell migration, a key step required for angiogenesis, is lower than that measured for endostatin and angiostatin, two other naturally occurring inhibitors of angiogenisis, making it one of the most potent endogenous inhibitors identified to date. While an understanding of the mechanism is not necessary to use the present invention and the present invention is not limited to any particular mechanism, it is contemplated that PEDF may inhibit angiogenesis by inducing microvascular endothelial cell apoptosis (Stellmach et al., Proc. Natl. Acad. Sci. 98:2593).

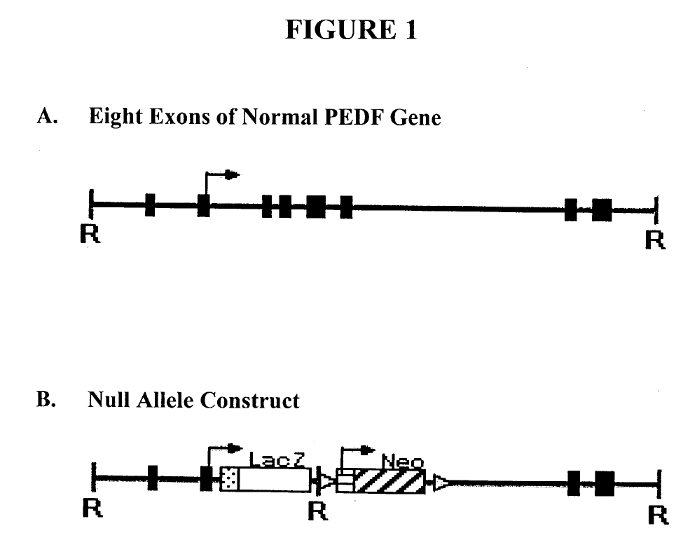

Mice were engineered to be null for PEDF using SV129 ES cells injected into C57B16 blastocysts. Standard methods may be used to generate the mice. In particular, a null allele construct disrupted the PEDF gene with an IRES-LacZ-Neo cassette between a 4.9 kb 5′-arm and a 3.7 kb 3′-arm (FIG. 1). Chimeric animals were obtained and a male chimeric mouse was mated to C57B16 females to obtain mice heterozygous for the PEDF null allele. Mice heterozygous for the null allele were mated to generate mice homozygous for the null allele of PEDF. The null mice are viable and fertile.

The null animals showed abnormalities in multiple systems including the prostate, neural retina, kidney vasculature, and cerebellar granule cells.

Prostates of 5-month old PEDF +/+ (wild type) and PEDF +/− mice were formalin fixed, paraffin-embedded, sectioned and stained with hemotoxylin and eosin. Unlike wildtype controls, there was epithelial cell hyperplasia with stratification of nuclei, moderate nuclear pleomorphism and an increased nuclear to cytoplasmic ratio in prostates of mice heterozygous for the null allele. The histological changes are similar to that observed in benign prostatic hyperplasia in humans. The prostates of 3-month old mice homozygous for the null allele have a more sever hyperplasia than that observed in the PEDF +/− mouse prostate glands. The prostate epithelial cells show a higher proliferative index. Also, the stroma shows a change in microvascular density.

These animals also show a disturbance in the ganglia cells of the retina with layering of retinal photoreceptors. The optic nerve is also smaller than wild-type animals. The animals further show a drop-out in cerebellar granule cells. The animals also show a change in kidney vasculature with an approximately 2-fold higher than baseline secretion of VEG-F.

In two hormone-sensitive organs, the pancreas and prostate, distinct stromal-epithelial phenotypes were evident. On gross examination, the PEDF^{-/-} pancreas (3 month old) appeared enlarged, expanding well beyond the midline. Microscopically, the acinar epithelial cells appeared less well-differentiated with increased nuclear to cytoplasmic ratios and cytologic atypia. The pancreatic blood vessels were excessive in number and dilated (Table 1). The exocrine glands were more abundant with many cells appearing to make only abortive attempts at true glandular structures with lumens. This growth disturbance was confirmed by a 3.8-fold increase in PCNA-positive epithelial cells in PEDF^{-/-} pancreas when compared to wildtype controls (8.33±0.54 vs. 2.21±0.28, P<0.001).

As noted above, it was also observed that PEDF^{-/-} mice developed prostatic hyperplasia. In striking contrast to PEDF^{+/+} prostates, where a simple columnar epithelial layer lined most glands, there was pronounced epithelial cell hyperplasia with stratification of the nuclei in 3-month-old PEDF^{-/-} prostates. Hyperplasia was also evident in PEDF^{+/-} but less pronounced. By 6-months, PEDF^{+/-} mice also had moderate nuclear pleomorphism and hyperchromatic nuclei, suggesting this was a progressive phenotype. Confirming a defect in growth control, the number of PCNA-positive epithelial cells was significantly higher in PEDF^{-/-} and PEDF^{+/-} prostates as compared to PEDF^{+/+}. Even when adjusted for total cell numbers, the increase remained significant. Because androgens regulate prostate development and growth, we measured serum testosterone levels and found no significant differences between PEDF^{+/+} (11.35±1.3 ng/ml; n=5) and PEDF^{+/-} (15.5±5.1; n=3, P=0.36) or PEDF^{-/-} (17.4±5.7; n=6, P 0.43) mice or between PEDF^{+/-} and PEDF^{-/-} (P=0.86), suggesting that the hyperplasia was not due to altered testosterone levels.

The pathologic changes within PEDF^{-/-} prostates were not confined to the epithelial cells. The scant numbers of stromal cells typical of wildtype controls were replaced by stroma with higher cellularity and increased MVD. The stromal MVD was 3.2 and 1.9 fold higher in PEDF^{-/-} and PEDF^{+/-} prostates as compared to PEDF^{+/+} (Table 1). The vessels in the null animals were also more dilated and muscular in PEDF^{-/-} mice with occasional intra-glandular vessels, a feature never seen in control prostates. While the present invention is not limited to a particular mechanism, the collected data suggest that dysregulated angiogenesis may be one mechanism underlying the hyperplastic epithelial growth observed in PEDF-deficient mice.

TABLE 1
Angiogenic phenotype in PEDF^{-/-} mouse tissues.
Microvessel Density

| Tissue | PEDF^{+/+} | PEDF^{-/-} | P-value |
|---|---|---|---|
| Retina* | 1.33 ± 0.3 | 7.0 ± 1.5 | 0.02 |
| Kidney | 50.6 ± 2.2 | 71.7 ± 3.5 | <0.05 |
| Pancreas | 3.87 ± 0.35 | 10.2 ± 0.45 | <0.0001 |
| Prostate | 4.87 ± 0.3528 | 15.5 ± 1.3 | <0.005 |

*Vessels were counted within the retina.

The null animals of the present invention find use as disease models screening compounds and characterizing the molecular basis of disease. For example, retinal phenotypes described above allows the animals to be used to screen for compound that find use in the treatment of diabetic retinopathy. Animals are given hypoxic injuries and are tested with a battery of drugs to determine which drugs can ameliorate the injury. In another example, based on the prostate phenotypes described above for the prostate epithelial tissue and stroma, the animal finds use for identifying drugs that diminish hyperplasia in a tissue-specific manner.

In view of the observed phenotypes, the null animals of the present invention find use for understanding and characterizing a number of diseases, conditions, and biological processes, including, but not limited to, developmental abnormalities (e.g., of the retina), blindness (e.g., Leber's Congenital Amaurosis), hypoxic injuries and responses, tumor growth, hormonal responses (e.g., androgen responses), macular degeneration (See e.g., Rasmussen et al., Hum. Gene. Ther. 12:2029 [2001]), and vascularization. In preferred embodiments, the animals of the present invention are employed to screen for anti-cancer compounds (e.g. to find compounds for treating cancer of the pacrease, prostate, kidney, eye, and bladder). A number of general screening utilities are provided below.

In some embodiments, the null animals of the present invention, or cells derived from the animals, find use as positive controls to evaluate the efficacy of PEDF inhibitors and to identify disease conditions that can be treated with PEDF inhibitors. In a screening assay to identify and assess the efficacy of PEDF inhibitors, a wild type animal (or cells derived therefrom) not treated with the inhibitor is used as the 0% inhibition standard, an animal heterozygous for an PEDF gene disruption (or cells derived therefrom) is used as the 50% inhibition standard and an animal homozygous for an PEDF gene disruption (or cells derived therefrom) is used as the 100% inhibition standard. The amount of PEDF activity in a subject treated with a PEDF inhibitor is then assessed relative to these standards.

Additionally, the animals of the invention are useful for determining whether a particular disease condition involves the action of PEDF and thus can be treated by a PEDF inhibitor. For example, an attempt can be made to induce a disease condition in an animal of the invention having a functionally disrupted PEDF gene. Subsequently, the susceptibility or resistance of the animal to the disease condition can be determined. A disease condition that is treatable with a PEDF inhibitor can be identified based upon resistance of an animal of the invention to the disease condition.

Through use of the subject transgenic animals or cells derived therefrom, one can identify ligands or substrates that bind to, modulate, antagonize or agonize PEDF. Screening to determine drugs that lack effect on PEDF is also of interest. Of particular interest are screening assays for agents that have a low toxicity for human cells. A wide variety of assays may be used for this purpose, including in vivo studies, determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cell derived therefrom. Cells may be freshly isolated from an animal, or may be immortalized in culture.

The term “agent” or “compound” as used herein describes any molecule, e.g. protein or pharmaceutical, with the capability of affecting the biological action of, for example, PEDF. Generally a plurality of assay mixtures are run in parallel with different agent concentrations to obtain a differential response to the various concentrations. Typically, one of these concentrations serves as a negative control, i.e. at zero concentration or below the level of detection.

Candidate agents (compounds) encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Screening may be directed to known pharmacologically active compounds and chemical analogs thereof.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening.

Antibodies specific for PEDF receptor polymorphisms may be used in screening immunoassays, particularly to detect the binding of substrates to PEDF, or to confirm the absence or presence of a PEDF in a cell or sample. Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. The cells may be dissociated, in the case of solid tissues, or tissue sections may be analyzed. Alternatively a lysate of the cells may be prepared.

For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

In some embodiments, the PEDF animals of the present invention are crossed with other transgenic models or other stains of animals to generate additional disease models. In another embodiment, a disease condition is induced by breeding an animal of the invention with another animal genetically prone to a particular disease. For example, in some embodiments, the PEDF mouse is bred with p27 knockout animals (Fero, et al. Cell 85:733 [1996]), PTEN knockout animals, p53 knockout animals (Donehower et al., Nature 356:215 [1992]), thrombospondin knockout animals (Lawler et al., J. Clin. Invest. 101:982 [1998]), etc. to generate animal models of neurological disorders and angiogenesis dependent diseases such as diabetic retinopathy, macular degeneration, rheumatoid arthritis, and solid benign and malignant tumors.

In some embodiments, the PEDF null animals are used to generate animals with an active PEDF gene from another species (a “heterologous” PEDF gene). In preferred embodiments, the gene from another species is a human gene. In some embodiments, the human gene is transiently expressed. In other embodiments, the human gene is stably expressed (e.g., the PEDF null animals are used to generate animals that are transgenic for human PEDF). Such animals find use to identify agents that inhibit human PEDF in vivo. For example, a stimulus that induces production of PEDF is administered to the animal in the presence and absence of an agent to be tested and the response in the animal is measured. An agent that inhibits human PEDF in vivo is identified based upon a decreased response in the presence of the agent compared to the response in the absence of the agent.

The animals of the invention, or cells derived therefrom, also can be used to screen PEDF inhibitors for side effects or toxicity resulting from the inhibitor's action on a target(s) other than PEDF itself (e.g., a PEDF isoforms). For example, a PEDF inhibitor is administered to an animal of the invention homozygous for a PEDF null mutation and the resulting effects are monitored to evaluate side effects or toxicity of the inhibitor. Since the animal lacks the normal target of the PEDF inhibitor (i.e., active PEDF protein), an effect observed upon administration of the inhibitor to the PEDF null mutant is attributed to a side effect of the PEDF inhibitor on another target(s) (e.g., a PEDF isoform). Accordingly, the animals of the invention are useful for distinguishing these side effects from the direct effects of the inhibitor on PEDF activity.

The animals of the invention, or cells derived therefrom, also find use to identify and/or clone PEDF homologues or isoforms in the absence of the normal PEDF background. The animals of the invention can also be used as recipients of tissues transplanted from wild-type animals to identify a tissue(s) that expresses a PEDF homologue or PEDF isoform having a detectable activity. The PEDF homologue or isoform so identified can be isolated from the tissue and/or cloned by standard molecular biology techniques.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A transgenic knockout mouse whose genome comprises a homozygous or heterozygous disruption in its endogenous pigment epithelium-derived factor gene, wherein said homozygous disruption prevents the expression of a functional pigment eptithelium-derived factor protein, and wherein said heterozygous disruption reduces the expression of functional pigment eptithelium-derived factor protein in said knockout mouse.

2. The mouse of claim 1, wherein said mouse exhibits hyperplasia of the prostate.

3. The mouse of claim 1, wherein said mouse exhibits increased microvascular density in the prostate.

4. The mouse of claim 1, wherein said mouse exhibits an enlarged pancreas.

5. The method of claim 1, wherein said mouse exhibit increased microvascular density in the pacrease.

6. The method of claim 1, wherein said mouse comprises a tumor.

7. The method of claim 6, wherein said tumor comprises pacreatic, prostatic, or bladder cells.

8. A method of screening for a compound, comprising:
A) exposing the transgenic mouse of claim 1 to said compound;
B) determining a response of said transgenic mouse to said compound, wherein a change in response compared to a transgenic mouse of claim 1 not exposed to said compound, indicates said response to said compound.

9. The method of claim 8, wherein said determining comprises examining prostate or pancreatic tissue from said mouse.

10. The method of claim 8, wherein said determining comprises evaluating prostate or pancreatic tissue from said mouse for increased microvascular density.

11. The method of claim 8, where said compound is suspected of being an anti-angiogenic compound.

12. The method of claim 8, wherein said compound is a known anti-angiogenic compound.

13. The method of claim 8, wherein said compound is a known carcinogen.

14. The method of claim 8, wherein said compound is suspected of being carcinogenic.

15. The method of claim 8, wherein said mouse comprises a tumor, wherein said tumor comprises pancreatic, prostatic or bladder cells.

16. A method for inducing cancer in a transgenic knockout mouse, comprising;
a) providing a transgenic knockout mouse whose genome comprises a homozygous or heterozygous disruption in its endogenous pigment epithelium-derived factor gene, wherein said homozygous disruption prevents the expression of a functional pigment eptithelium-derived factor protein, and wherein said heterozygous disruption reduces the expression of functional pigment eptithelium-derived factor protein in said knockout mouse; and
b) exposing said transgenic knockout mouse to conditions such that cancer formation is promoted in said transgenic knockout mouse.

17. The method of claim 16, wherein said conditions comprise administering a carcinogen to said transgenic knockout mouse.

18. The method of claim 16, wherein said conditions comprise injecting cancerous cells into said transgenic knockout mouse.

19. The method of claim 16, wherein said conditions comprise exposing said transgenic knockout mouse to radiation.

20. The method of claim 16, wherein said conditions comprise exposing said transgenic knockout mouse to biological agents known to induce cancer.
